(19) United States
(12) Patent Application Publication
(10) Pub. No.: US 2011/0236949 A1
(43) Pub. Date: Sep. 29, 2011

(54) Methods for Processing Biological Tissues

(75) Inventors: E. Christopher Orton, Fort Collins, CO (US); Shiori Arai, Columbus, OH (US); Carla Maria Ribeiro Lacerda, Fort Collins, CO (US); Leigh Gareth Griffiths, Davis, CA (US)

(73) Assignee: COLORADO STATE UNIVERSITY RESEARCH FOUNDATION, Fort Collins, CO (US)

(21) Appl. No.: 12/888,119

(22) Filed: Sep. 22, 2010

Related U.S. Application Data
(60) Provisional application No. 61/249,488, filed on Oct. 7, 2009.
(60) Provisional application No. 61/244,651, filed on Sep. 22, 2009.

(51) Int. Cl. C12N 13/00

(52) U.S. Cl. 435/173.9

(57) ABSTRACT

Methods are provided for removing or separating the cellular and/or soluble macromolecular component of a biological tissue from the extracellular matrix component of the biological tissue, comprising embedding the biological tissue in an electrically conductive semi-solid or solid supporting medium and applying an electric field to the tissue-medium complex. Additionally, methods are provided for decellularizing a skin fragment.

### RELATED APPLICATIONS AND INCORPORATIONS BY REFERENCE

This application claims priority to U.S. Provisional Patent Application Ser. No. 61/244,651, filed on Sep. 22, 2009, and to U.S. Provisional Patent Application Ser. No. 61/249,488, filed on Oct. 7, 2009, which are incorporated herein by reference in their entirety.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

### TECHNICAL FIELD

This invention relates to a method(s) for removing cells (i.e. decellularization), soluble proteins, antigens (i.e. antigen removal), phospholipids, carbohydrates, nucleic acids, and other macromolecules from biological tissues, including organs, for the purpose of removing or separating the cellular and/or soluble macromolecular component of a tissue/organ from the scaffold, extracellular matrix, and/or insoluble component of a tissue/organ. The method described herein involves embedding a tissue or organ in an electrically conductive semi-solid or solid supporting medium (e.g. agarose gel) and applying an electric field to the resulting tissue-gel complex for the purpose of causing soluble proteins, protein-detergent complexes, nucleic acids, and other macromolecules to migrate or move out of the tissue along an electrical potential gradient.

### BACKGROUND

Bioprosthetic heart valves, currently used to replace defective heart valves in humans, are constructed from animal (xenogeneic) tissues such as porcine aortic valves or bovine pericardium. Bioprosthetic heart valves are treated with gluteraldehyde and other chemicals to “fix” the cellular component and cross-link the matrix or scaffold component of the tissue. Such treatment prevents severe acute immune rejection of the tissue that would otherwise quickly destroy the implanted bioprosthesis. It is, however, now clear that both humoral and cell-mediated chronic immune rejection of bioprosthetic heart valves occurs despite glutaraldehyde-fixation. (Human and Zilla (2001) J Long Term Eff Med Implants 11(3-4):199-220, “Inflammatory and immune processes: the neglected villain of bioprosthetic degeneration?”; Dahm, et al. (1990) J Thorac Cardiovasc Surg 99(6):1082-1090, “Factor S M, Frater R W. Immunogenicity of gluteraldehyde-tanned bovine pericardium”; Dahm, et al. (1995) Ann Thorac Surg 60(2 Suppl):5348-352, “Relevance of immunologic reactions for tissue failure of bioprosthetic heart valves”; Human and Zilla (2001) Ann Thorac Surg 71(5 Suppl):5385-388, “Characterization of the immune response to valve bioprostheses and its role in primary tissue failure”; Mirzaie, et al. (2000) Scand Cardiovasc J 34(6):589-592, “Influence of gluteraldehyde fixation on the detection of SLA-I and II antigens and calcification tendency in porcine cardiac tissue”; Salgaller and Bajpai (1985) J Biomed Mater Res 19(1):1-12, “Immunogenicity of gluteraldehyde-treated bovine pericardial tissue xenografts in rabbits”; Manji, et al. (2006) Circulation 114(4):318-327, “Glutaraldehyde-fixed bioprosthetic heart valve conduits calcify and fail from xenograft rejection”). Mounting evidence implicates chronic antibody formation and immune rejection in bioprosthetic heart valve degeneration and calcification. (Dahm, et al. (1990), supra.; Dahm, et al. (1995), supra.; Salgaller and Bajpai (1985), supra.; Manji, et al. (2006) supra.)

Tissue-engineering is a scientific field devoted to the creation of “living” tissue or organ replacements. One general approach to tissue-engineering involves the creation of “biological tissue scaffolds” through the removal of native cells from animal (xenogeneic) tissues by treatments or processes termed “decellularization”. An implicit assumption of tissue decellularization is that antigens that would otherwise incite acute or chronic immune tissue rejection are largely associated with the cellular component of the tissue and are, thus, removed by decellularization treatments. After decellularization, a living functional tissue is created by replacing the cellular component of the tissue with cells from the recipient patient (e.g. autogenous adult stem cells) or cells from the another human source (e.g. allogeneic umbilical cells) by processes known as “recellularization”. Alternatively, a decellularized tissue can be implanted without prior recellularization. Such tissues can be used as a biomaterial for reconstructive surgeries or procedures, or to enhance or direct regenerative processes. Several physical and chemical methods for decellularization of cardiovascular tissues including porcine heart valves, porcine small diameter vessels, bovine pericardium, and human allografts have been reported. (Steinhoff, et al. (2000) Circulation 102 [suppl III]: III-50-55, “Tissue engineering of pulmonary heart valves on allogeneic acellular matrix conduits: in vivo restoration of valve tissue”; Cebotari, et al. (2002) Circulation 106 [suppl I]: I-63-1-68, “Construction of autologous human heart valve based on an acellular allograft matrix”; Bertipaglia, et al. (2003) Ann Thorac Surg 75:1274-1282. “Cell characterization of porcine aortic valve and decellularized leaflets repopulated with aortic valve interstitial cells: the VESALO project”; Schenke-Layland, et al. (2003) Cardiovasc Res 60:497-509, “Complete dynamic repopulation of decellularized heart valves by application of defined physical signals—an in vitro study”; Orton, U.S. Pat. No. 5,192,312, issued Mar. 9, 1993, entitled “Treated Tissue for Implantation and Methods of Treatment and Use”; Elkins, et al. (2001) Ann Thorac Surg 71:S428-432, “Decellularized human valve allografts”; Curtil, et al. (1997) Cryobiology 34:13-22, “Freeze drying of cardiac valves in preparation for cellular repopulation”; Malone, et al. (1984) J Vasc Surg 1:181-191, “Detergent-extracted small-diameter vascular prostheses”; Wilson, et al. (1990) Trans ASAIO 36:M340-343, “Acellular matrix allograft small caliber vascular prostheses”; Courtman, et al. (1994) J Biomed Mater Res 28:655-666, “Development of a pericardial acellular matrix biomaterial: biomechanical and mechanical effects of cell extraction”; Wilson, et al. (1995) Ann Thorac Surg 60:S353-358, “Acellular matrix: a biomaterials approach for coronary artery bypass and heart valve replacement”; Vesley, et al. (1995) Ann Thorac Surg 60:S359-364, “The hybrid xenograft/autograft bioprosthetic heart valve: in vivo evaluation of tissue extraction”; O'Brien, et al. (1999) Sem Thorac Cardiovasc Surg 11:194-200, “The Synergraft valve: A new acellular (non-gluteraldehyde-fixed) tissue heart valve for autologous recellularization first experimental studies before clinical implantation”; Goldstein, et al. (2000) Ann Thorac Surg 70:1962-1969, “Transpecies heart valve transplant: advanced studies of a bioengineered xeno-autograft”; Courtman, et al. (1991) Trans Soc Biomaterials 16:62, “Development of a pericardial acellular matrix bioprostheses:effects of cellular extraction on mechanics and morphology”; Booth, et al. (2002) J Heart Valve Dis 11:457-462, “Tissue engineering of cardiac valve prostheses I: Development and histological characterization of an acellular porcine scaffold”; Korossis, et al. (2002) J Heart Valve Dis 11:463-471, “Tissue engineering of cardiac valve prostheses II: Biochemical characterization of decellularized porcine aortic heart valves”; Bader, et al. (1998) Eur J Cardiothorac Surg 14:279-284, “Tissue engineering of heart valves—human endothelial cell seeding of detergent acellularized porcine valves”) Physical methods for decellularization have included gamma radiation (Orton, U.S. Pat. No. 5,192,312, issued Mar. 9, 1993, entitled “Treated Tissue for Implantation and Methods of Treatment and Use”) and freeze drying. (Curtil, et al. (1997), supra.) Chemical methods for decellularization have included combinations of hypotonic lysis, (Elkins, et al. (2001), supra.; Courtman, et al. (1994), supra.; Wilson, et al. (1995), supra.; O'Brien, et al. (1999), supra.; Goldstein, et al. (2000), supra.) detergents, (Bertipaglia, et al. (2003), supra.; Schenke-Layland, et al. (2003), supra.; Malone, et al. (1984), supra.; Wilson, et al. (1990), supra.; Courtman, et al. (1994), supra.; Wilson, et al. (1995), supra.; Vesley, et al. (1995), supra.; Courtman, et al. (1991), supra.; Booth, et al. (2002), supra.; Bader, et al. (1998), supra.) trypsin, (Steinhoff, et al. (2000), supra.; Cebotari, et al. (2002). supra.) and nucleases. (Elkins, et al. (2001), supra.; O'Brien, et al. (1999), supra.; Goldstein, et al. (2000), supra.; Courtman, et al. (1991), supra.; Kasimir, et al. (2003) Int J Artif Organs 26:421-427, “Comparison of different decellularization procedures of porcine heart valves”) Currently favored, recently published, and commercially available decellularization treatments employ combinations of hypotonic cell lysis and treatment with ionic and/or nonionic detergents including sodium dodecyl sulfate (SDS), sodium deoxycholate, and/or Triton-X. (Seebacher, et al. (2008) Artif Organs 32(1): 28-35, “Biomechanical properties of decellularized porcine pulmonary valve conduits”; Liao, et al. (2008) Biomaterials 29(8): 1065-1074, “Effects of decellularization on the mechanical and structural properties of the porcine aortic valve leaflet”; Tudorache, et al. (2007) J Heart Valve Dis 16(5): 567-73, “Tissue engineering of heart valves: biomechanical and morphological properties of decellularized heart valves”; Oswal, et al. (2007) J Heart Valve Dis 16(2): 165-74, “Biomechanical characterization of decellularized and cross-linked bovine pericardium”; Iwai, et al. (2007) J Artif Organs 10(1): 29-35, “Minimally immunogenic decellularized porcine valve provides in situ recellularization as a stentless bioprosthetic valve”)

Current decellularization treatments likely do not completely remove antigens from tissues and, more importantly, do not completely mitigate immune rejection of tissue-engineered tissues. (Gonçalves, et al. (2005) J Heart Valve Dis 14:212-217, “Decellularization of bovine pericardium for tissue-engineering: antigen removal and recellularization”; Simon, et al. (2003) Eur J Cardio-Thorac Surg 23:1002-6, “Early failure of the tissue engineered porcine heart valve SYNERGRAFT in pediatric patients”) Indeed, a recent study reveals that current detergent-based decellularization methods do not completely remove detectable antigens from treated porcine aortic valves and bovine pericardium. (Arai and Orton (2009) J Heart Valve Dis 18:439-443, “Immunoblot detection of soluble protein antigens from sodium dodecyl sulfate- and sodium deoxycholate-treated candidate bioscaffold tissues”) Additional recent investigations indicate that treatment of tissues with high concentrations of detergents renders the tissue scaffold incompatible or poorly compatible with subsequent recellularization, which is likely a consequence of residual detergent in the tissue. (Rieder, et al. (2004) J Thoracic Cardiovasc Surg 127:399-405, “Decellularization protocols of porcine heart valves differ importantly in efficiency of cell removal and susceptibility of the matrix recellularization with human vascular cells”; Caamaño, et al. (2009) J Heart Valve Dis 18:101-105, “Does sodium dodecyl sulfate wash out of detergent-treated bovine pericardium at cytotoxic concentrations?”)

The present invention is directed toward overcoming one or more of the problems discussed above.

### BRIEF SUMMARY OF THE INVENTION

Decellularized tissues can be recellularized naturally after implantation, decellularized tissues can be subjected to methods to enhance recellularization after implantation, or decellularized tissues can be scaffolds for reconstructive surgery or making bioprostheses. Ideally, tissue decellularization (antigen removal) treatments completely remove cells and/or antigens, retain physical and biomechanical properties of the scaffold, and are compatible with subsequent recellularization. Indeed, the performance of bioprosthetic heart valves may be improved by the removal of cells or soluble macromolecules that elicit an immune response.

The methods disclosed herein enhance decellularization and antigen removal from biological tissues, including organs, for example, allograft (human) and xenograft (animal) tissues, prior to implantation in humans and, thereby, decrease immune response to these tissues. State-of-the-art treatments employ combinations of physical treatments (e.g. radiation, mechanical pressure), treatment with hypotonic or hypertonic salts, treatment with ionic, nonionic, Zwitterionic detergents including sodium dodecyl sulfate (SDS), sodium deoxycholate, CHAPS and/or Triton-X, enzymatic treatments (e.g. trypsin, endonucleases, exonucleases), buffers, protease inhibitors, and passive aqueous washout. Because these treatments do not appear to completely remove antigens and, likely, leave behind residual detergent in the tissue, tissue compatibility is frequently compromised to a non-negligible extent. The methods described herein, thus, constitute an improvement over other techniques, as they result in enhanced decellularization and antigen removal and should, for example, decrease immune rejection of the treated graft tissues.

The methods of the invention allow for the removal of cellular and/or soluble macromolecules from a biological tissue via decellularization without damaging the structural integrity of the tissue.

The methods are based on semi-solid or solid-phase electrophoresis and offer an improvement over liquid-based decellularization techniques. These improvements include migration of soluble proteins, antigens, nucleic acids, macromolecules, surfactants and other molecules from the biological tissue into the electrically conductive semi-solid or solid supporting medium. Embedding the biological tissue into, for example, agarose gel immobilizes the tissue and allows for efficient and directional application of an electrical current to the tissue, causing directed migration of the cellular and/or soluble macromolecular component in the electrical field and out of the immobilized tissue.

Whereas macromolecules migrate strictly according to charge in liquid-based electrophoresis, they migrate according to charge and molecular weight (size) in a solid supporting medium-based electrophoresis system. The orientation of the tissue in the medium can be optimized and maintained relative to the direction of current application. The migration of soluble macromolecules into the supporting medium can be measured and monitored. Molecules that migrate into the supporting medium are not immediately available to diffuse back into tissue.

Current parameters can be further optimized to minimize alterations of biomechanical properties of the tissue. Where a biological tissue placed in a liquid solution in an electric field will be subject to change in shape, the structural integrity and shape of a biological tissue embedded in a semi-solid or solid supporting medium will be maintained. The structural integrity constitutes the capacity of the tissue matrix to withstand forces such as tension, compression, shear, flexure, and support. The maintenance of this integrity and shape is especially important if the tissue is to perform a load-bearing function, such as a cardiovascular or orthopedic device. Maintaining the structural integrity of the biological tissue is critical, for example, for tissues more than one layer of cells thick, for example, a heart valve conduit or a piece of lung.

Thus, in one aspect, the invention provides a method for removing or separating the cellular and/or soluble macromolecular component of a biological tissue from the extracellular matrix component of the biological tissue, comprising embedding the biological tissue in an electrically conductive semi-solid or solid supporting medium and applying an electric field to the resulting tissue-medium complex.

In another aspect, the invention provides a method for removing or separating the cellular and/or soluble macromolecular component of a biological tissue from the extracellular matrix component of the biological tissue, comprising embedding the biological tissue in an electrically conductive semi-solid or solid supporting medium and applying an electric field to the resulting tissue-medium complex, thus removing or separating the cellular and/or soluble macromolecular component of the biological tissue from the extracellular matrix component of the biological tissue.

In one embodiment of a method of the invention, the application of the electric field does not result in a substantial change in the biomechanical properties of the tissue.

In one embodiment of a method of the invention, the cellular component comprises native cells of the biological tissue. In another embodiment of a method of the invention, the macromolecular component comprises at least one of soluble proteins, antigens, phospholipids, carbohydrates, and nucleic acids of the biological tissue.

In one embodiment of a method of the invention, the application of the electric field causes charged soluble proteins, protein-detergent complexes, nucleic acids, or other macromolecules to migrate or move out of the tissue and into the surrounding supporting medium along an electrical potential gradient.

In another embodiment of a method of the invention, the biological tissue is a tissue, an organ, a bioprosthesis, a biomaterial, a xenogeneic tissue (xenograft), an allogeneic tissue (allograft), or a tissue-engineered tissue. In still another embodiment of a method of the invention, the biological tissue is selected from the group consisting of heart valve, vessel, vascular conduit, artery, vein, skin, dermis, pericardium, dura, intestine, intestinal submucosa, ligament, tendon, bone, cartilage, muscle, ureter, urinary bladder, liver, lung, and heart. In yet another embodiment of a method of the invention, the biological tissue is dermis.

In one embodiment of a method of the invention, the orientation of the biological tissue is optimized and maintained relative to the direction of application of electric field/current.

In another embodiment of a method of the invention, the electric field is applied across the long axis of the tissue. The long axis of the tissue can, for example, be the longest axis, i.e., end to end. In still another embodiment of a method of the invention, the electric field is applied across the short axis of the tissue. The short axis of the tissue can, for example, be the shortest axis, i.e., top to bottom. In yet another embodiment of a method of the invention, the electric field is applied radially from the inside to the outside of a spherical, cylindrical, or complex 3-dimensional hollow tissue. The latter is, for example, contemplated as an option for vascular conduits.

In one embodiment of a method of the invention, the removal and/or separation of the cellular component of the biological tissue is followed by recellularization.

In another embodiment of a method of the invention, the biological tissue is derived from a mammal. The mammal can, for example, be a human.

In another embodiment of a method of the invention, the biological tissue is treated with an ionic detergent prior to and/or during the application of the electric field. In still another embodiment of a method of the invention, the ionic detergent is sodium dodecyl sulfate (SDS). In still another embodiment of a method of the invention, the biological tissue is treated with about 0.01% to about 5% detergent prior to and/or during the application of the electric field. In still another embodiment of a method of the invention, the biological tissue is treated in order to effect a change in the pH of the tissue.

In one embodiment of a method of the invention, the electric field is applied to the tissue-medium complex over a period of about 1.5 hours to about 24 hours. In another embodiment of a method of the invention, the electric field is applied to the tissue-medium complex over a period of about 4 hours to about 24 hours. In still another embodiment of a method of the invention, the electric field applied results in an electrical potential between about 1 and about 100 V. In still another embodiment of a method of the invention, the electric field applied results in an electrical potential between about 10 and about 100 V.

In one embodiment of a method of the invention, the supporting medium is an agarose gel. In another embodiment of a method of the invention, the agarose gel is a single density gel comprising agarose between about 0.5 and about 2% (w/v).

In another embodiment of a method of the invention, the electric field applied constitutes a current between about 0.1 and about 1 Amp. In still another embodiment of a method of the invention, the composition of the supporting medium is manipulated to optimize the electrical conductive properties of the tissue-medium complex. In yet another embodiment of a method of the invention, the composition of the supporting medium is manipulated to selectively remove a desired macromolecular component according to electric charge and molecular weight.

In one aspect, the invention provides a method for processing a biological tissue prior to implantation or transplantation, comprising embedding the biological tissue in an electrically conductive semi-solid or solid supporting medium and applying an electric field to the resulting tissue-medium complex.

In another aspect, the invention provides a method for processing a biological tissue prior to implantation or transplantation, comprising embedding the biological tissue in an electrically conductive semi-solid or solid supporting medium and applying an electric field to the resulting tissue-medium complex, thus processing the biological tissue prior to implantation or transplantation.

In one embodiment of a method of the invention, the processing is followed by recellularization.

In another embodiment of a method of the invention, the biological tissue is derived from a mammal. The mammal can, for example, be a human.

In one embodiment of a method of the invention, the biological tissue is treated with an ionic detergent prior to and/or during the application of the electric field. In another embodiment of a method of the invention, the biological tissue is treated with about 0.01% to about 5% detergent prior to and/or during the application of the electric field. In still another embodiment of a method of the invention, the biological tissue is treated in order to effect a change in the pH of the tissue.

In one embodiment of a method of the invention, the electric field is applied to the tissue-medium complex over a period of about 1.5 hours to about 24 hours. In another embodiment of a method of the invention, the electric field is applied to the tissue-medium complex over a period of about 4 hours to about 24 hours.

In one aspect, the invention provides a method for preparing a biological tissue scaffold, comprising embedding a biological tissue in an electrically conductive semi-solid or solid supporting medium and applying an electric field to the resulting tissue-medium complex.

In one embodiment of a method of the invention, the scaffold is for use in reconstructive surgeries or procedures or for use in the enhancement or direction of regenerative processes.

In another aspect, the invention provides a method for decellularizing a skin fragment, comprising the steps of: treating the skin fragment in a hypertonic solution; embedding the skin fragment in an electrically conductive semi-solid or solid supporting medium; subjecting the resulting skin fragment-medium complex to electrophoresis; and washing the skin fragment in a hypotonic or isotonic solution.

In still another aspect, the invention provides a method for decellularizing a skin fragment, comprising the steps of: treating the skin fragment in a hypertonic solution; embedding the skin fragment in an electrically conductive semi-solid or solid supporting medium; subjecting the resulting skin fragment-medium complex to electrophoresis; and washing the skin fragment in a hypotonic or isotonic solution, thus decellularizing the skin fragment.

In one embodiment, the method of the invention further comprises the step of treating the skin fragment with an ionic detergent. In another embodiment of a method of the invention, the skin fragment-medium complex is subjected to electrophoresis across the short axis of the fragment. The short axis of the tissue can, for example, be the shortest axis, i.e., top to bottom.

In one embodiment of a method of the invention, the electrophoresis causes charged soluble proteins, protein-detergent complexes, nucleic acids, or other macromolecules to migrate or move out of the fragment and into the surrounding supporting medium along an electrical potential gradient. In another embodiment of a method of the invention, the decellularization of the skin fragment is followed by recellularization. In still another embodiment of a method of the invention, the skin fragment is derived from a mammal. The mammal can, for example, be a human.

In one embodiment of a method of the invention, the ionic detergent is sodium dodecyl sulfate (SDS). In another embodiment of a method of the invention, the concentration of ionic detergent is between about 0.01 and about 5%.

In another embodiment of a method of the invention, the skin fragment is treated in order to effect a change in the pH of the fragment. In yet another embodiment of a method of the invention, the skin fragment-medium complex is subjected to electrophoresis over a period of about 1.5 to about 24 hours. In still another embodiment of a method of the invention, the skin fragment-medium complex is subjected to electrophoresis over a period of about 4 to about 24 hours.

In one embodiment of a method of the invention, the skin fragment-medium complex is subjected to electrophoresis at an electrical potential of between about 1 and about 100 V. In another embodiment of a method of the invention, the skin fragment-medium complex is subjected to electrophoresis at an electrical potential of between about 10 and about 100 V.

In one embodiment of a method of the invention, the supporting medium is an agarose gel. In another embodiment of a method of the invention, the agarose gel is a single density gel comprising agarose between about 0.5 and about 2% (w/v). In still another embodiment of a method of the invention, the skin fragment-medium complex is subjected to electrophoresis at a current between about 0.1 and about 1 Amp.

In one embodiment of a method of the invention, the composition of the supporting medium is manipulated to optimize the electrical conductive properties of the skin fragment-medium complex. In another embodiment of a method of the invention, the composition of the supporting medium is manipulated to selectively remove a desired macromolecular component according to electric charge and molecular weight.

In one embodiment of a method of the invention, the skin fragment is washed in the hypotonic or isotonic solution for about 12 to about 96 hours. In another embodiment of the invention, the isotonic solution is phosphate-buffered saline. In still another embodiment of a method of the invention, the hypotonic solution is Tris-buffered water. In yet another embodiment of a method of the invention, the skin fragment is treated in the hypertonic solution for about 15 to about 36 hours. In still another embodiment of a method of the invention, the hypertonic solution is about 0.5 to about 1.5M sodium chloride.

Other aspects of the invention are described in or are obvious from the following disclosure and are within the ambit of the invention.

### BRIEF DESCRIPTION OF THE DRAWINGS

The following Detailed Description of the Invention, given by way of Examples, but not intended to limit the invention to specific embodiments described, may be understood in conjunction with the accompanying figures, in which:

FIG. 1 shows, above, the results of immunoblot (SDS-PAGE) analysis of soluble proteins extracted from porcine aortic valve conduit after sequential treatment with hypotonic lysis, sodium dodecyl sulfate (SDS) (0.25% or 1%) for 24 hours, and tissue-gel electrophoresis (TE) at 0V, 60V, and 120 V for four hours without aqueous washout. Data show a decrease in extractable soluble protein in tissue-gel electrophoresis-treated valve compared to control valve; and below, in bar graph form, the relative optical density of porcine aortic valve conduit extract immune banding expressed as percent of optical density of untreated control (UT) after sequential treatment with hypotonic lysis, sodium dodecyl sulfate (SDS) (0.25% or 1%) for 24 hours, and tissue-gel electrophoresis (TE) at 0V, 60V, and 120 V for four hours without aqueous washout. Data are mean±S.D. (n=3 gel replicates for each treatment). Asterisks (*) over horizontal bars indicate significant difference (p<0.05) between treatments.

FIG. 2 shows, in bar graph form, the relative optical density (O.D.) of porcine aortic valve conduit extract immune banding expressed as percent of optical density of untreated control after sequential treatment with hypotonic lysis, sodium dodecyl sulfate (SDS) for 24 hrs, with and without tissue-gel electrophoresis (TE) at 120 V for 4 hrs, and passive aqueous washout for 96 hrs. Data are mean±S.D. (n=3 each treatment). By two-way ANOVA, both SDS concentration (p=0.001) and TE (p=0.025) enhanced antigen removal from porcine aortic valve (PAV).

FIG. 3 shows, above, the results of immunoblot analysis of soluble protein antigens extracted from bovine pericardium after sequential treatment with hypotonic lysis, sodium dodecyl sulfate (SDS) (0.25% or 1%) for 24 hrs, solid-phase tissue electrophoresis (TE) at 0V, 60V, and 120V for 4 hrs; and below, in bar graph form, the relative optical density (O.D.) of bovine pericardium extract immune banding expressed as percent of optical density of untreated control (UT) after sequential treatment with hypotonic lysis, sodium dodecyl sulfate (SDS) (0.25% or 1%) for 24 hrs, tissue-gel electrophoresis (TE) at 0V, 60V, and 120 V for 4 hrs. Data are mean±S.D. (n=3). Asteriks (*) over horizontal bars indicate significant difference (p<0.05) between treatments. Molecular weight (MW) is shown as kD.

FIG. 4A shows the results of tissue-gel electrophoresis-decreased cellularity of human dermis compared to control-treated human dermis; histological hematoxylin and eosin staining of human dermis treated with or without (control) tissue-gel electrophoresis at 350 mA (10 V). Control sample treated with 0 current and voltage. 100× magnification. FIG. 4B shows the results of SDS-PAGE analysis of human dermis treated with or without (control) tissue-gel electrophoresis at 350 mA (10 V). Control sample treated with zero current and voltage. Data show a decrease in extractable soluble protein in tissue-gel electrophoresis-treated dermis compared to control-treated dermis.

### DETAILED DESCRIPTION

### Definitions

The term “biological tissue” as used herein refers to a collection of interconnected cells and extracellular matrix that perform a similar function or functions within an organism. Biological tissues include, without limitation, connective tissue, muscle tissue, nervous tissue (of the brain, spinal cord, and nerves), epithelial tissue, and organ tissue. Connective tissue includes fibrous tissue like fascia, tendon, ligaments, heart valves, bone, and cartilage. Muscle tissue includes skeletal muscle tissue, smooth muscle tissue, such as esophageal, stomach, intestinal, bronchial, uterine, urethral, bladder, and blood vessel tissue, and cardiac muscle tissue. Epithelial tissue includes simple epithelial tissue, such as alveolar epithelial tissue, blood vessel endothelial tissue, and heart mesothelial tissue, and stratified epithelial tissue.

The term “conductive solid supporting medium” as used herein refers to a material that has an initial liquid phase that allows embedment of a tissue or organ, and then becomes a solid through a process of solidification (e.g. agarose) or polymerization (e.g. acrylamide) while retaining its electrically conductive properties. The term “semi-solid supporting medium” refers to a medium that behaves like a solid when it is not under pressure, but can be forced to flow under conditions of pressure. After electrophoresis, a solid supporting medium can be separated from the tissue by manually breaking or using other physical means to break apart the solid medium. Alternatively, the solid supporting medium can be separated from the tissue by converting it back into a liquid-phase using chemical (e.g. enzymes or de-polymerizing agents) or physical (e.g. temperature) means. After electrophoresis, a semi-solid supporting medium can be separated from the tissue by subjecting it to pressure and causing it to flow.

The biological tissue can additionally be selected, without limitation, from the group consisting of heart valve, vessel, vascular conduit, artery, vein, skin, dermis, pericardium, dura, intestinal submucosa, ligament, tendon, bone, cartilage, ureter, urinary bladder, liver, lung, umbilical cord, and heart. Multiple tissues/tissue types comprise organs. Organs are included herein under the term “biological tissue”.

The term “organ” as used herein refers to a collection of tissues joined in a structural unit to serve a common function.

The term “dermis” as used herein refers to the layer of skin between the epidermis and the subcutaneous tissues.

The term “epithelial tissue” as used herein refers to the tissue covering the whole surface of the body or lining certain organ systems exposed to the external environment, such as the gastrointestinal tract, the urogenital tract, or the lung. It is made up of cells closely packed and arranged in at least one layer. This tissue is specialized to form a covering or lining of all internal and external body surfaces.

The term “subject” as used herein refers to a vertebrate, preferably a mammal. Mammals include, without limitation, humans, primates, wild animals, feral animals, farm animals (porcine, bovine, ovine sheep, equine), sports animals, and pets.

The term “cellular and/or soluble macromolecular component” as used herein refers to soluble substances constituting portions of the cell or produced by cells, including cell membranes, cytosol, and soluble macromolecules (e.g. proteins, nucleic acids, polypeptides, glycoproteins, carbohydrates, lipids, phospholipids, etc.).

The term “recellularization” as used herein refers to removing the cells from a biological tissue, for example, an organ, leaving only the extracellular matrix to be subsequently repopulated with cells. Recellularization is especially useful in tissue engineering.

The term “extracellular matrix” (ECM) as used herein refers to the extensive and complex structure between the cells—the extracellular part of the biological tissue. The ECM generally comprises the structural component of the tissue, including its organization, shape, and strength (i.e., ability to resist external forces). Due to its diverse nature and composition, the ECM can serve many additional functions, such as providing support and anchorage for cells, segregating tissues from one another, and regulating intercellular communication. The ECM can influence a cell's dynamic behavior. In addition, it sequesters a wide range of cellular growth factors and acts as a local depot for them. Included in the ECM are insoluble structural molecules that have been secreted by cells and comprise components such as collagen, elastin, and large soluble proteoglycans.

The terms “comprises”, “comprising”, are intended to have the broad meaning ascribed to them in U.S. Patent Law and can mean “includes”, “including” and the like. Moreover, the use of the term “including”, as well as other forms, such as “includes” and “included”, is not limiting.

The invention can be understood more fully by reference to the following detailed description and illustrative examples, which are intended to exemplify non-limiting embodiments of the invention.

### Additional Embodiments of the Invention

Methods of the invention involve embedding a biological tissue (including an organ) in an electrically conductive semi-solid or solid supporting medium (for example, a gel) and applying an electrical field for the purpose of causing soluble proteins and other macromolecules to migrate, move, or diffuse out of the tissue along an electrical gradient.

The tissue or organ may be derived from any number of living subjects, e.g. human, mammal, or animal.

The supporting medium may, for example, be any sort of gel (e.g. agarose; polyacrylamide; or other gel-forming agents familiar to the ordinarily skilled artisan) or quasi-solid state or solid-state material that exhibits ionic conductivity and is sufficiently porous so as to allow migration of charged macromolecules through the material under an applied electric field. In most cases, a gel is a polymer whose composition, conductivity, and porosity are manipulated or changed to encourage/discourage migration of macromolecules based on charge and/or molecular weight. For example, if effecting the migration out of proteins or small nucleic acids, including viral nucleic acids, the supporting medium can be composed of different concentrations of agarose. The concentration of agarose prior to solidification may, without limitation, be between about 0.5 to about 2% (w/v). The solution in which the agarose gel is mixed may or may not include an ionic detergent such as SDS.

The gel can, for example, be a single density gel. The gel might also, for example, be a gradient gel with a percent agarose ranging from between about 3-5% and about 10-12% (w/v).

Electrophoresis refers to the electromotive force used to move the molecules out of the biological tissue and into the electrically conductive semi-solid or solid supporting medium. The strength and duration of the electric field may vary—depending on the properties of the supporting medium, the cellular and/or soluble macromolecular component to be removed, the dimensions of the biological tissue, the interelectrode distance, the amount of applied current, etc. The applied potential may be anywhere from a few volts to several hundred volts.

The voltage actually running through the biological tissue can vary as a result of the orientation of the tissue in the electric field. In one embodiment, the biological tissue may be embedded in the supporting medium so that the electric field is applied across the long axis of the tissue (i.e., end-to-end). In another embodiment, the biological tissue may be embedded in the supporting medium so that the electric field is applied across the short axis of the tissue (i.e., top-to-bottom). It is additionally contemplated, in one embodiment, that the electric field is applied radially from the inside (lumen) to the outside of a spherical, cylindrical, or complex 3-dimensional tissue (e.g., lung). In other words, any angle is contemplated for the application of the electric field, where the tissue is viewed as 3-dimensional, with an X, Y, and Z axis.

With such manipulation of the orientation of the biological tissue within the electric field (for example, applying the electric field across the shortest axis of the tissue), the inventors have found that a significantly lower (for example, 10-fold lower) voltage is generated for the same current. This can alleviate possible concerns of tissue denaturation due to high temperatures potentially associated with higher voltages.

As mentioned above, the applied potential may be anywhere from a few volts to several hundred volts. However, in an additional embodiment of the invention, the voltage could be between about 10 Volts and about 100 Volts for dermis, if the current is applied across the short axis of the embedded tissue. Biological tissues of a similar thickness to dermis (for example, human dermis) can, in still another embodiment, be subjected to the same voltage range, i.e., between about 10 Volts and about 100 Volts, if the electrical field is oriented to the shortest axis, for example, thickness of the tissue. However, a thicker tissue, for example, cardiac muscle, might require subjection to a different voltage range. The same would apply for a tissue wherein the electrical field is not oriented across the shortest axis.

Current to be applied to the biological tissue embedded in the supporting medium likewise ranges based on multiple factors, including thickness of the biological tissue and orientation of the same within the electric field. In one embodiment, for example, the current applied to dermis is between about 0.1 Amp and about 1 Amp.

Additional parameters that can be varied in embodiments of the processing of the invention include the temperature, for example, between about 4° C. and about room temperature (about 24° C.). The ratio of volume of biological tissue to volume of gel can, in one embodiment of the invention, be between about 1:5 to about 1:20 tissue:gel. This range may, of course, vary with thickness of biological tissue and/or orientation of the tissue within the electrical field.

Proteins, unlike nucleic acids, can have varying net charges, complex shapes, and molecular weights. Therefore, they may not migrate into the semi-solid or solid supporting medium (for example, gel) at similar rates, or at all, when placing a negative to positive electromotive force on the biological tissue. Therefore, the tissue may be treated beforehand, for example, with SDS. Prior or concurrent treatment of the biological tissue with SDS, another ionic detergent, or some other chemical imparts a uniform charge to soluble proteins and protein antigens within the tissue, causing them to move out of the tissue into the surrounding medium when the tissue is placed in an electric field. Detergents contemplated for such use include, without limitation, sodium dodecyl sulfate, sodium dodecyl sulfonate, polyethylene glycol-containing detergent, and sodium dodecyl sarcosinate.

In one embodiment of the invention, the SDS concentration is from about 0.01% to about 5%; in another embodiment, about 0.1% to about 1%. The inventors have found that SDS leaching from treated biological tissue can be cytotoxic (Caamano, S., et al. 2009 J Heart Valve Dis 18(1):101-105; Rieder, E., et al. 2004 J Thorac Cardiovasc Surg 127:399-405).

A method according to the invention can be employed as sole treatment to accomplish tissue decellularization and antigen removal, or it can be an adjunct to other decellularization or antigen removal methods. An additional benefit of this treatment method is that it could decrease or eliminate residual ionic detergent or other chemical from the tissue scaffold that impairs subsequent tissue recellularization, as mentioned above. Other native macromolecules such as phospholipids or nucleic acids that contribute to undesirable degenerative and chemical processes in implanted tissues (e.g. tissue calcification) may also be removed by the method described here. Lastly, infectious agents or particles (e.g. viral nucleic acids) could be removed by the method described here.

Decellularization renders the biological tissue “substantially acellular”. “Substantially acellular” signifies having at least about 50% less, preferably at least about 55% less, more preferably at least about 60% less, more preferably at least about 65% less, more preferably at least about 70% less cellular material associated with the cells than the natural or living state of the biological tissue. Degree of acellularity can, for example, be assessed by light microscopy using standard histology techniques, electron microscopy (including transmission electron microscopy (TEM) and scanning electron microscopy (SEM)), as well as solubilization of the tissue, followed by protein determination using standard techniques.

Decellularization may be followed by rinsing or a period of passive washout (for example, in phosphate buffered saline), sterilization (for example, using alcohol, antibiotic, radiation), and/or storage (for example, using buffered storage solution and/or freezing).

Additional Applications of the Invention

In specific embodiments, the invention may have application in processing and implantation of gluteraldehyde-fixed, chemically-treated, or unfixed bioprostheses, biomaterials, or xenogeneic tissues (xenografts) including heart valves, vascular conduits, arteries, veins, skin, dermis, ligaments, tendons, bone, cartilage, muscle, ureter, urinary bladder, liver, heart, and other organs; or processing and transplantation of fresh, preserved, or banked allogeneic tissues (allografts) including vessels, vascular conduits, arteries, veins heart valves, skin, dermis ligaments, tendons, bone, cartilage, muscle, ureter, urinary bladder, liver, heart, or other organs; or the development of natural biological matrices for tissue-engineered tissues and organs including heart valves, vessels, skin, dermis, ligaments, bone, cartilage, muscle, ureter, urinary bladder, liver, heart, or other organs.

In additional embodiments, tissue constructs may be used as implants, tissue fillers, burn dressings, wound dressings, blood vessel grafts, blood vessel replacements, and the like. Medical graft materials of the invention can be used in the repair or reconstruction of tissues such as nervous tissue, dermal tissue (ex: in wound care), cardiovascular tissue (including vascular and cardiac), pericardial tissue, muscle tissue, bladder tissue, ocular tissue, periodontal tissue, bone, connective tissue (tendons, ligaments), and the like. Medical graft materials of the invention may also be used in conjunction with one or more secondary components to construct a medical device (ex: a balloon-expandable or self-expanding stent).

In further embodiments, tissue constructs can be treated with agents such as growth factors and/or pharmaceuticals. Growth factors may, for example, be used to promote recellularization, vascularization, or epithelialization. Antibodies or antibiotics may be used to prevent potential infection from implant. Matrix components may also be used. Other so-called recellularization agents include, without limitation, chemoattractants, cytokines, chemokines, and derivatives thereof.

In one embodiment, decellularized human dermis can be recellularized prior to implantation by co-culturing the tissue processed according to a method of the invention with autogenous adult mesenchymal stem cells (e.g., bone marrow-derived mesenchymal stem cells) or autogenous epidermal stem cells. In vitro co-culture conditions can, in specific embodiments, be under static conditions for cell culture or can take place in a bio-reactor mimicking certain desired in vivo conditions. As mentioned above, processed tissue can, in specific embodiments, be treated with growth factors (e.g., basic fibroblast growth factor) or chemokines to enhance cellular ingrowth/migration into the tissue and/or to direct cells to adopt appropriate phenotypes. Such treatments could be employed to enhance in vitro recellularization before implantation of the processed biological tissue or to enhance in vivo recellularization after implantation of the tissue, and would be familiar to the ordinarily skilled artisan.

Another application of a method according to the invention is to mitigate or prevent immune rejection of transplanted tissues/organs between individuals within a species (i.e., allografts) or between species (i.e., xenografts) by the removal of antigens from the tissue/organ. Yet another application of a method according to the invention is to mitigate or prevent destructive or degenerative chemical processes (e.g. tissue calcification) in tissues/organs after implantation. Still another application of a method according to the invention is the removal of infectious agents (e.g. viral nucleic acids) from tissues prior to implantation or transplantation. Another application of a method according to the invention is to pursue enhanced uniformity of biological, immunological, and biomechanical properties between batches of treated tissues.

The invention described here involves the treatment of allogeneic (i.e., derived from within the same species) and xenogeneic (i.e., derived from different species) tissues for the purpose of removing cells, cellular debris, antigens, proteins, nucleic acids, phospholipids, and other macromolecules prior to implantation. Some proteins, nucleic acids, phospholipids and other molecules have a natural net positive or negative ionic charge that causes them to move or migrate in an electrical field. As mentioned above, proteins (and other macromolecules, for that matter) can, however, additionally be treated with the ionic detergent, such as sodium dodecyl sulfate (SDS), which strongly binds to soluble proteins and imparts a net uniform charge to proteins that is proportional to their size (i.e., molecular weight).

Kits

In one aspect, the invention provides kits for removing or separating the cellular and/or soluble macromolecular component of a biological tissue from the extracellular matrix component of the biological tissue, comprising an electrically conductive semi-solid or solid supporting medium and a unit for application of an electric field and instructions for use of the medium and unit in conjunction with the biological tissue.

A kit of the invention can further comprise detergent(s). For example, the kit can include SDS for treatment of the biological tissue before or after application of the electric field. A kit of the invention can additionally comprise materials and associated instructions for analysis of the tissue after application of the electric field. A kit of the invention can also comprise materials for storage of the biological tissue after application of the electric field.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

### EXAMPLES

The following examples are provided for illustrative purposes only and are not intended to limit the scope of the invention.

### Example 1

Bovine pericardium was obtained from cadavers and transported to the lab in the sterilized transfer/washing solution consisting of phosphate buffered saline (PBS) with 10 KIU/ml aprotinin (Sigma Aldrich), 0.1% EDTA (Fluka), 1% antibiotic solution (Sigma). Bovine pericardium was cleaned from adipose tissues, cut into about 1.5 cm² squares, embedded into one of two types of gel: 12% polyacrylamide gel or 2% agarose gel. Size of the final tissue-gel complex was 8.6×6.8 cm. Polyacrylamide gel was prepared with 12% bis-acrylamide solution (Bio-Rad), 0.375M Tris HCl buffer pH 8.8 (Sigma), 0.1% (w/v) SDS (Bio-Rad), 0.1% (w/v) ammonium persulfate (Sigma) and 0.004% (v/v) TEMED (Bio-Rad). Agarose gel was made by heating and mixing 0.1% SDS (Bio-Rad) and 2% agarose (Sigma) in tris-borate-EDTA buffer pH 8.3 (Sigma). Bovine pericardium was placed on a spacer glass plate and bound with a short glass plate. Tissue was placed on upper half. 12% polyacrylamide or 2% agarose gel was then poured into the space between the two plates. The tissue-gel complexes were left to solidify at room temperature for 1 hour and then stored at 4° C. for overnight.

The polyacrylamide tissue-gels were placed on Biorad Mini-Protean 3 Cell (Bio-Rad) and run for 20 hours at 250V constant. Agarose tissue-gels were run for 4 hours at 125V constant. Current was between 0.01 to 0.02 A for both experiments. Sham tissue-gels were created for each type of gel and treated in an identical manner except that electrical current was not applied to the gel. After electrophoresis, the gel was stained with silver to detect soluble proteins in the tissue and gel surrounding the tissues.

In the polyacrylamide tissue-gel, silver staining indicated that proteins were moved out from the tissues into the gel by the electric current (data not shown). In sham polyacrylamide tissue-gel, there was no evidence that proteins migrated from the tissue. In the agarose tissue-gel, heavy protein staining was evident at the bottom of the gel toward the positive electrode (cathode) indicating that negatively charged proteins had been drawn out of the tissue and into the supporting medium (data not shown). The sham agarose tissue-gel showed some migration of proteins in all directions, including simple diffusion. The instant example shows the utility of electrophoretically removing protein from a biological tissue embedded in a supporting medium using embodiments of the present invention.

### Example 2

Porcine aortic valves with associated aorta were obtained postmortem from healthy animals, transported under aseptic conditions in phosphate buffered saline (PBS) with 10 KIU/ml aprotinin, 0.1% EDTA, 1% antibiotic solution (100 U/ml penicillin, 100 μg/ml streptomycin and 0.25 μg/ml amphotericin B), and stored at −80° C. until used. Porcine aortic valve conduits (PAV) were sectioned prior to treatment. Each section contained a single aortic valve leaflet and its associated aortic wall. Tissue treatments were performed at room temperature under agitation. Tissues were subjected to 4 h of hypotonic cell lysis treatment by immersion in deionized water with aprotinin 10 KIU/ml, 6.5% (v/v) Tris-buffer, 0.1% EDTA and 1% antibiotics. Tissues were then treated overnight with sodium dodecyl sulfate (SDS) in PBS at concentrations ranging from 0.01% to 1%.

PAV were embedded in 2% agarose. Concentration of SDS in gel and running buffer were matched to the concentration used to treat the tissue prior to tissue electrophoresis. Solidified gels containing tissues were placed in a horizontal gel electrophoresis unit (Bio-Rad) in Tris-Glycine SDS running buffer run for up to 12 h at 0 V, 60 V, or 120 V at 4° C. Finally, tissues were removed from gels and washed in PBS with aprotinin 10 KIU/ml, 0.1% EDTA and 1% antibiotics for 96 h with complete change of the washing solution every 24 h.

Immunoblot assay for soluble protein antigens was performed on treated and untreated PAV as previously described (Arai, S., et al. 2009 J Heart Valve Dis 18:439-443). Briefly, immune serum was generated by injecting homogenized PAV subcutaneously into New Zealand white rabbits every 2 weeks. Serum was collected after 70 days and stored at −80° C. until used. Soluble proteins were extracted from PAV for immunoblot assay. Equal wet weights of PAV were minced, placed in a solution of 0.1% SDS, 10 mM Tris HCl (pH 8.0), 100 KIU/ml aprotinin, 1 mM dithiotheritol, 2 mM MgCl₂ 10 mM KCl and 0.5 mM pefabloc. Tissue suspensions were shaken on ice for 1 h and centrifuged at 17,000 g for 20 min. The supernatant was saved. The pellet was re-suspended in a solution of 1.25% SDS, 10 mM Tris HCl (pH 8.0), 100 KIU/ml aprotinin, 1 mM dithiotheritol, 2 mM MgCl₂ 10 mM KCl and 0.5 mM pefabloc, shaken on ice for 1 h, and centrifuged at 17,000 g for 20 min. The supernatants were combined and concentrated using Amicon (Millipore) for 30 min at 7,500 g and saved. Protein fractions were mixed with an equal volume of sample buffer (Invitrogen) and reduced in 5% (v/v) β-mercaptoethanol (Gibco) at 95° C. for 3 min. Equal volumes of protein solution were loaded on 4-12% 1.5 mm Tris-glycine gels (Invitrogen). Gels were run for 1.5 h at a constant 125 V at room temperature.

Proteins were transferred to 0.2 μm pore size nitrocellulose membranes (Invitrogen) at a constant 25V at room temperature for 1.5 h. Membranes were washed for 5 minutes in Tris-buffered saline with 1% Tween20, blocked with 5% BSA for 1 hour at room temperature, and left at 4° C. overnight. Membranes were incubated with rabbit anti-PAV immune serum for 2 h at room temperature on a shaker using 1:1000 titer. Membranes were washed with Tris-buffered saline with 1% Tween 20, incubated with horseradish peroxidase-conjugated swine anti-rabbit IgG (DAKO). Membranes were washed again with Tris-buffered saline with 1% Tween 20. Signals were developed by Supersignal West Pico/Femto (6:1) chemiluminescent substrate (Pierce) using Hyperfilm ECL (Amersham Biosciences). Three replicate gels were run for each protein extraction. Optical density of immune banding was measured using Adobe Photoshop software (version 7.0). Antigen removal was expressed as a relative optical density (%) determined by dividing the immune banding optical density of treated tissues by the optical density of untreated control tissues. Results were analyzed by one-way or two-way ANOVA. Values of p<0.05 were considered significant.

Treated and untreated PAV were fixed with buffered 10% formaldehyde solutions overnight, dehydrated, and embedded in paraffin. Sections were sectioned, stained with hematoxylin and eosin, and examined for cellularity and the morphology of extracellular matrix.

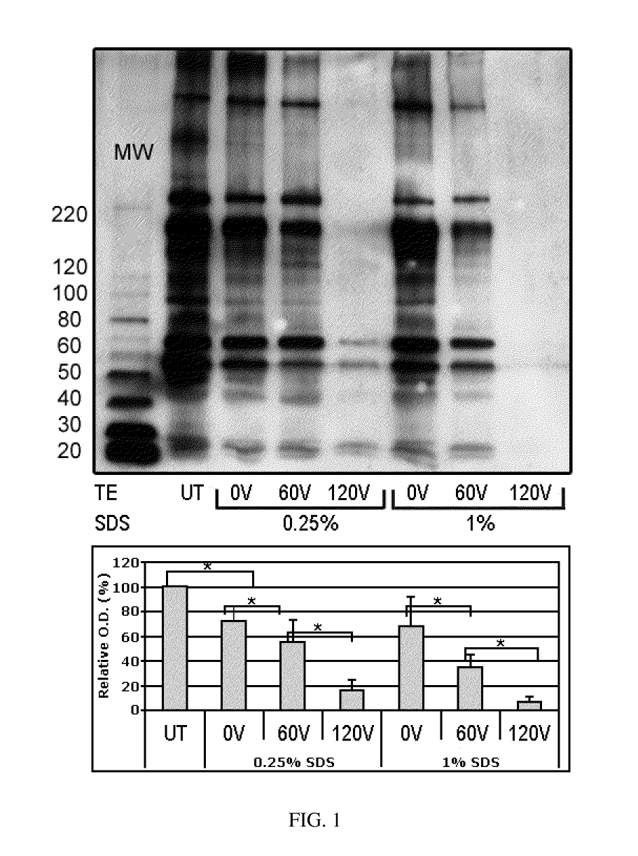

The effect of tissue-gel electrophoresis (TE) at three voltages (0, 60, & 120 V) and two SDS concentrations (0.25% & 1.0%) on antigen removal from PAV before aqueous washout are shown in FIG. 1. All treatments increased (p<0.05) protein antigen removal compared to untreated control tissue. Voltage increased (p<0.0001) protein removal from PAV. Antigen removal was not different between 0.25% and 1.0% SDS (p=0.37) before aqueous washout. Antigen removal from PAV treated with 0.25% SDS and 120 V before aqueous washout was not different between 4, 8, and 12 hrs of TE running time.

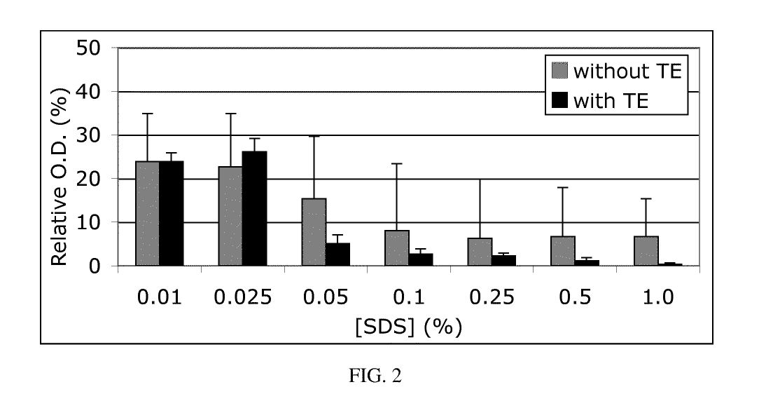

The effect of SDS concentration on antigen removal from PAV with and without TE at 120 V for 4 h followed by 96 h aqueous washout is shown in FIG. 2. SDS concentration (p=0.001) and TE (p=0.025) in dependently enhanced antigen removal from PAV based on two-way ANOVA. Only treatment of PAV with TE and 1.0% SDS resulted in no detection of protein antigens.

Histology was studied of the aortic wall portion of untreated PAV and PAV treated with 0.05% or 1.0% SDS with or without TE at 250 V for four hours (followed by 96 hours washout). Treatment with 1.0% SDS with TE resulted in apparent complete acellularity of the aortic conduit wall (data not shown). TE also enhanced removal of nucleic acids from tissues.

### Example 3

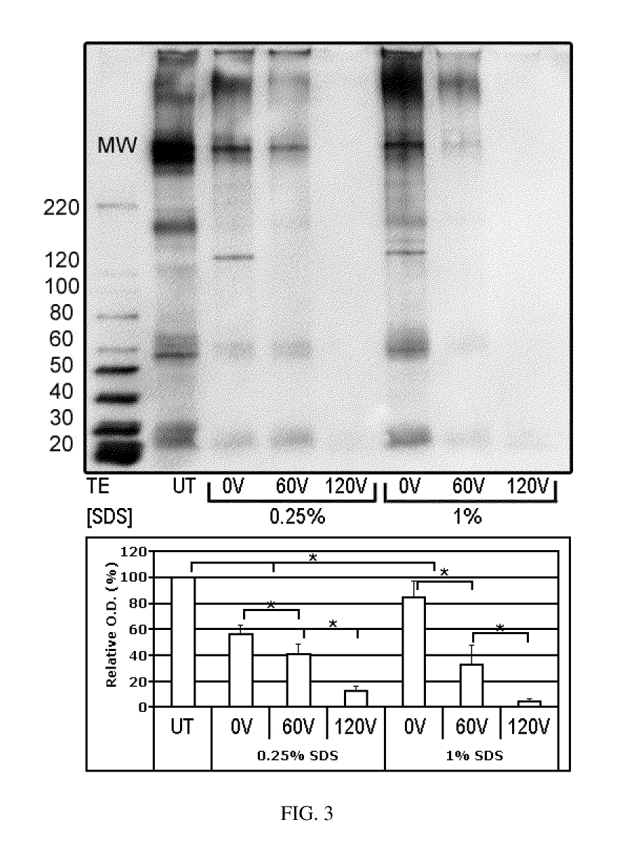

Bovine pericardium was subjected to sequential treatment as follows: hypotonic lysis, SDS (0.25 or 1%) for 24 hrs, and solid-phase TE at 0V, 60V, and 120V for four hrs (similar to above-described experiments). Immunoblot analysis showed decreasing amounts of soluble protein antigens with increasing voltage (and somewhat more markedly for 1% SDS) (data not shown). Relative optical densities are provided in FIG. 3.

### Example 4

In order to demonstrate the feasibility and efficacy of tissue-gel electrophoresis as a decellularization method for human dermis, full thickness human skin was pre-treated for 24 h with a sterile hypertonic solution containing 0.5% SDS (w/v) at room temperature. The composition of the hypertonic solution was 1M sodium chloride, 10 mM Tris pH 7.6, 1 mM EDTA disodium salt, with protease inhibitors, antibiotics and antimycotics. After this treatment, epidermis was removed by gentile scrapping with a scalpel blade.

Dermis was subsequently embedded in 1.5% agarose gel made with electrophoresis buffer. The composition of the buffer and gel was 40 mM Tris base, 40 mM acetic acid, 1 mM EDTA disodium salt. The tissue-gel was allowed to solidify for 1 hour. The tissue-gel was placed in the electrophoresis chamber. The electrophoresis chamber was filled with the afore-mentioned electrophoresis buffer, which covered the tissue-gel, as well as the cathode and anode. An electric field was applied across the short-axis of the tissue (side-to-side of chamber) for 6 h at a constant current of 350 mA (10 V) and at room temperature. A control sample was treated in an identical manner, except that the electrophoresis step was run with 0 current and voltage. After tissue-gel electrophoresis, dermis was removed from the gel and washed in hypotonic solution (identical to hypertonic solution described above, without 1 M sodium chloride) for 6 h.

After the full treatment, treated and control dermis samples were prepared for histology and protein analyses. A 3-mm strip was infiltrated with formalin and the remaining of the tissue was homogenized for protein extraction. Soluble proteins extracted from dermis and equal volumes of the protein extract solution were analyzed by SDS-PAGE.

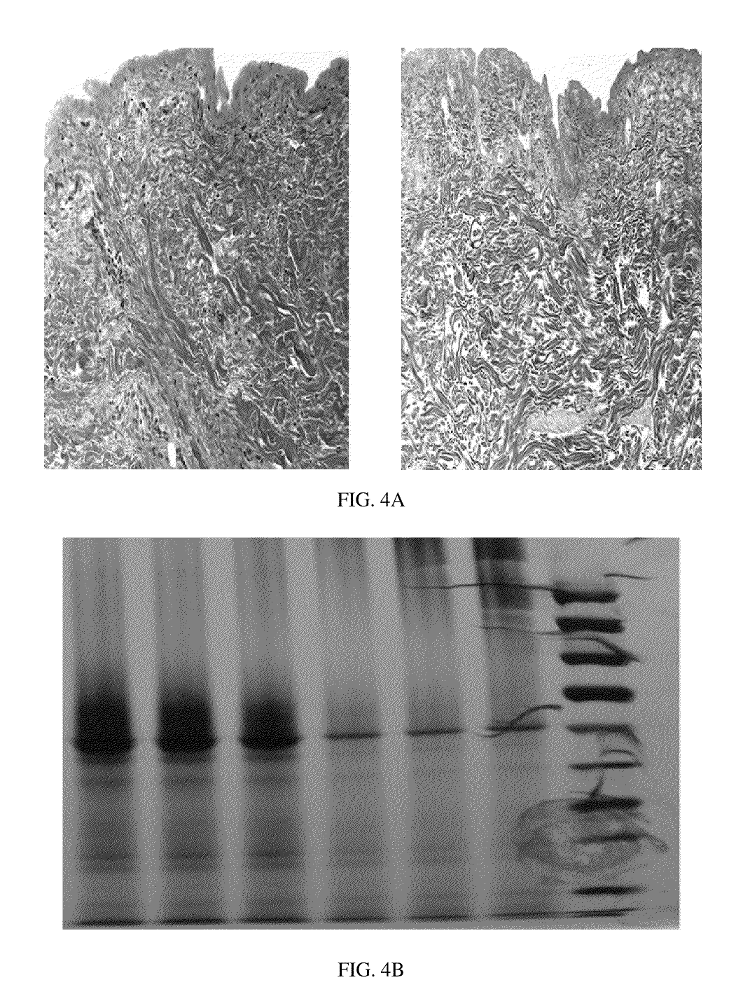

Tissue-gel electrophoresis decreased cellularity of human dermis compared to control-treated human dermis (FIG. 4A). SDS-PAGE analysis demonstrated a decrease in extractable soluble protein in tissue-gel electrophoresis-treated dermis compared to control-treated dermis (FIG. 4B).

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

What is claimed is:

1. A method for removing or separating the cellular and/or soluble macromolecular component of a biological tissue from the extracellular matrix component of the biological tissue, comprising embedding the biological tissue in an electrically conductive semi-solid or solid supporting medium and applying an electric field to the resulting tissue-medium complex.

2. The method of claim 1, wherein the cellular component comprises native cells of the biological tissue.

3. The method of claim 1, wherein the macromolecular component comprises at least one of soluble proteins, antigens, phospholipids, carbohydrates, and nucleic acids of the biological tissue.

4. The method of claim 1, wherein the application of the electric field causes charged soluble proteins, protein-detergent complexes, nucleic acids, or other macromolecules to migrate or move out of the tissue and into the surrounding supporting medium along an electrical potential gradient.

5. The method of claim 1, wherein the biological tissue is a tissue, an organ, a bioprosthesis, a biomaterial, a xenogeneic tissue (xenograft), an allogeneic tissue (allograft), or a tissue-engineered tissue.

6. The method of claim 1, wherein the biological tissue is selected from the group consisting of heart valve, vessel, vascular conduit, artery, vein, skin, dermis, pericardium, dura, intestine, intestinal submucosa, ligament, tendon, bone, cartilage, muscle, ureter, urinary bladder, liver, lung, and heart.

7. The method of claim 6, wherein the biological tissue is dermis.

8. The method of claim 1, wherein the orientation of the biological tissue is optimized and maintained relative to the direction of application of current.

9. The method of claim 1, wherein the electric field is applied across the long axis of the tissue.

10. The method of claim 1, wherein the electric field is applied across the short axis of the tissue.

11. The method of claim 1, wherein the electric field is applied radially from the inside to the outside of a spherical, cylindrical, or complex 3-dimensional hollow tissue.

12. The method of claim 1, wherein the removal and/or separation of the cellular component of the biological tissue is followed by recellularization.

13. The method of claim 1, wherein the biological tissue is derived from a mammal.

14. The method of claim 13, wherein the mammal is a human.

15. The method of claim 1, wherein the biological tissue is treated with an ionic detergent prior to and/or during the application of the electric field.

16. The method of claim 15, wherein the ionic detergent is sodium dodecyl sulfate (SDS).

17. The method of claim 15, wherein the biological tissue is treated with about 0.01% to about 5% detergent prior to and/or during the application of the electric field.

18. The method of claim 1, wherein the biological tissue is treated in order to effect a change in the pH of the tissue.

19. The method of claim 1, wherein the electric field is applied to the tissue-medium complex over a period of about 1.5 hours to about 24 hours.

20. The method of claim 19, wherein the electric field is applied to the tissue-medium complex over a period of about 4 hours to about 24 hours.

21. The method of claim 1, wherein the electric field applied results in an electrical potential between about 1 and about 100 V.

22. The method of claim 21, wherein the electric field applied results in an electrical potential between about 10 and about 100 V.

23. The method of claim 1, wherein the supporting medium is an agarose gel.

24. The method of claim 23, wherein the agarose gel is a single density gel comprising agarose between about 0.5 and about 2% (w/v).

25. The method of claim 1, wherein the electric field applied constitutes a current between about 0.1 and about 1 Amp.

26. The method of claim 1, wherein the composition of the supporting medium is manipulated to optimize the electrical conductive properties of the tissue-medium complex.

27. The method of claim 1, wherein the composition of the supporting medium is manipulated to selectively remove a desired macromolecular component according to electric charge and molecular weight.

28. A method for processing a biological tissue prior to implantation or transplantation, comprising embedding the biological tissue in an electrically conductive semi-solid or solid supporting medium and applying an electric field to the resulting tissue-medium complex.

29. The method of claim 28, wherein the processing is followed by recellularization.

30. The method of claim 28, wherein the biological tissue is derived from a mammal.

31. The method of claim 30, wherein the mammal is a human.

32. The method of claim 28, wherein the biological tissue is treated with an ionic detergent prior to and/or during the application of the electric field.

33. The method of claim 28, wherein the biological tissue is treated with about 0.01% to about 5% detergent prior to and/or during the application of the electric field.

34. The method of claim 32, wherein the biological tissue is treated in order to effect a change in the pH of the tissue.

35. The method of claim 28, wherein the electric field is applied to the tissue-medium complex over a period of about 1.5 hours to about 24 hours.

36. The method of claim 35, wherein the electric field is applied to the tissue-medium complex over a period of about 4 hours to about 24 hours.

37. A method for preparing a biological tissue scaffold, comprising embedding a biological tissue in an electrically conductive semi-solid or solid supporting medium and applying an electric field to the resulting tissue-medium complex.

38. The method of claim 37, wherein the scaffold is for use in reconstructive surgeries or procedures or for use in the enhancement or direction of regenerative processes.

39. A method for decellularizing a skin fragment, comprising the steps of:
i) treating the skin fragment in a hypertonic solution;
ii) embedding the skin fragment in an electrically conductive semi-solid or solid supporting medium;
iii) subjecting the resulting skin fragment-medium complex to electrophoresis; and
iv) washing the skin fragment in a hypotonic or isotonic solution.

40. The method of claim 39, further comprising the step of treating the skin fragment with an ionic detergent.

41. The method of claim 39, wherein the skin fragment-medium complex is subjected to electrophoresis across the short axis of the fragment.

42. The method of claim 39, wherein the electrophoresis causes charged soluble proteins, protein-detergent complexes, nucleic acids, or other macromolecules to migrate or move out of the fragment and into the surrounding supporting medium along an electrical potential gradient.

43. The method of claim 39, wherein the decellularization of the skin fragment is followed by recellularization.

44. The method of claim 39, wherein the skin fragment is derived from a mammal.

45. The method of claim 44, wherein the mammal is a human.

46. The method of claim 40, wherein the ionic detergent is sodium dodecyl sulfate (SDS).

47. The method of claim 40, wherein the concentration of ionic detergent is between about 0.01 and about 5%.

48. The method of claim 40, wherein the skin fragment is treated in order to effect a change in the pH of the fragment.

49. The method of claim 39, wherein the skin fragment-medium complex is subjected to electrophoresis over a period of about 1.5 to about 24 hours.

50. The method of claim 49, wherein the skin fragment-medium complex is subjected to electrophoresis over a period of about 4 to about 24 hours.

51. The method of claim 39, wherein the skin fragment-medium complex is subjected to electrophoresis at an electrical potential of between about 1 and about 100 V.

52. The method of claim 51, wherein the skin fragment-medium complex is subjected to electrophoresis at an electrical potential of between about 10 and about 100 V.

53. The method of claim 39, wherein the supporting medium is an agarose gel.

54. The method of claim 53, wherein the agarose gel is a single density gel comprising agarose between about 0.5 and about 2% (w/v).

55. The method of claim 39, wherein the skin fragment-medium complex is subjected to electrophoresis at a current between about 0.1 and about 1 Amp.

56. The method of claim 39, wherein the composition of the supporting medium is manipulated to optimize the electrical conductive properties of the skin fragment-medium complex.

57. The method of claim 39, wherein the composition of the supporting medium is manipulated to selectively remove a desired macromolecular component according to electric charge and molecular weight.

58. The method of claim 39, wherein the skin fragment is washed in the hypotonic or isotonic solution for about 12 to about 96 hours.

59. The method of claim 39, wherein the isotonic solution is phosphate-buffered saline.

60. The method of claim 39, wherein the hypotonic solution is Tris-buffered water.

61. The method of claim 39, wherein the skin fragment is treated in the hypertonic solution for about 15 to about 36 hours.

62. The method of claim 39, wherein the hypertonic solution is about 0.5 to about 1.5M sodium chloride.
